(19) United States
(12) Patent Application Publication
(10) Pub. No.: US 2015/0100011 A1
(43) Pub. Date: Apr. 9, 2015

(54) Development of Active Cells with a Stimulating Prosthesis

(71) Applicant: Cisco Technology, Inc., (US)

(72) Inventors: Shaun Ashwin Kumar, Rockdale (AU); Paul Michael Carter, West Pennant Hills (AU)

(21) Appl. No.: 14/048,350

(22) Filed: Oct. 8, 2013

(51) Int. Cl. A61N 5/06; A61N 1/32

(52) U.S. Cl. CPC A61N 5/062; A61N 1/327; A61N 2005/0643
USPC 604/20

(57) ABSTRACT

Presented herein are techniques that transform a recipient's cells into active cells. More specifically, the techniques presented herein transfect optically-sensitive elements into the cells. The optically-sensitive elements may cause the nerve cells to fire or activate (i.e., generate an action potential) in the presence of electromagnetic radiation, or may prevent the nerve cells from firing or activating in the presence of electromagnetic radiation.

### BACKGROUND

1. Field of the Invention

The present invention relates generally to stimulating prostheses, and more particularly, to the development of active cells with a stimulating prosthesis.

2. Related Art

Hearing loss, which may be due to many different causes, is generally of two types, conductive and/or sensorineural. Conductive hearing loss occurs when the normal mechanical pathways of the outer and/or middle ear are impeded, for example, by damage to the ossicular chain or ear canal. Sensorineural hearing loss occurs when there is damage to the inner ear, or to the nerve pathways from the inner ear to the brain.

Individuals who suffer from conductive hearing loss typically have some form of residual hearing because the hair cells in the cochlea are undamaged. As such, individuals suffering from conductive hearing loss typically receive an auditory prosthesis that generates motion of the cochlea fluid. Such auditory prostheses include, for example, acoustic hearing aids, bone conduction devices, and direct acoustic stimulators.

In many people who are profoundly deaf, however, the reason for their deafness is sensorineural hearing loss. Those suffering from some forms of sensorineural hearing loss are unable to derive suitable benefit from auditory prostheses that generate mechanical motion of the cochlea fluid. Such individuals can benefit from implantable auditory prostheses that stimulate nerve cells of the recipient's auditory system in other ways (e.g., electrical, optical and the like). Cochlear implants are often proposed when the sensorineural hearing loss is due to the absence or destruction of the cochlea hair cells, which transduce acoustic signals into nerve impulses. Auditory brainstem stimulators might also be proposed when a recipient experiences sensorineural hearing loss due to damage to the auditory nerve.

### SUMMARY

In one aspect of the invention, a stimulating prosthesis is provided. The stimulating prosthesis comprises a delivery mechanism configured to introduce optically-sensitive elements into a proximity of cells of a recipient; and a stimulating assembly comprising one or more electrical stimulating contacts configured to apply an electrical field to the cells of the recipient so such that the optically-sensitive elements are transfected into the cells during the electroporation of the cells.

In another aspect of the present invention, a method is provided. The method comprises introducing optically-sensitive elements into a proximity of a recipient's cells, and applying an electrical field to cells of the recipient with a stimulating prosthesis such that the optically-sensitive elements are transfected into the cells.

In a further aspect, a stimulating prosthesis is provided. The stimulating prosthesis comprises a delivery mechanism configured to introduce optically-sensitive elements into a proximity of a recipient's cells; and one or more stimulating contacts disposed in the recipient's cochlea configured to open pores in the recipient's cells such that the optically-sensitive elements are transfected into the cells.

### BRIEF DESCRIPTION OF THE DRAWINGS

Embodiments of the present invention are described herein in conjunction with the accompanying drawings, in which:

FIG. 1 is a schematic diagram of a cochlear implant configured to develop optically active nerve cells in accordance with embodiments presented herein;

FIG. 2 is a graph illustrating various phases of an idealized action potential as the potential passes through a nerve cell;

FIG. 3A is a schematic diagram illustrating a nerve cell prior to transformation of the nerve cell into an optically active nerve cell in accordance with embodiments presented herein;

FIG. 3B is a schematic diagram illustrating an enlarged view of a portion of the nerve cell of FIG. 3A;

FIG. 3C is a schematic diagram illustrating the nerve cell prior of FIG. 3A during transformation of the nerve cell into an optically active nerve cell;

FIG. 3D is a schematic diagram illustrating an enlarged view of a portion of the nerve cell of FIG. 3C;

FIG. 3E is a schematic diagram illustrating the nerve cell prior of FIG. 3A after transformation of the nerve cell into an optically active nerve cell;

FIG. 3F is a schematic diagram illustrating an enlarged view of a portion of the nerve cell of FIG. 3E;

FIG. 4A is a schematic diagram illustrating an enlarged view of a portion of the nerve cell of FIG. 3E prior to exposure to electromagnetic radiation

FIG. 4B is a schematic diagram illustrating an enlarged view of a portion of the nerve cell of FIG. 3E during exposure to electromagnetic radiation;

FIG. 5 is a side view of an implantable component of a cochlear implant configured to develop optically active nerve cells in accordance with embodiments presented herein;

FIGS. 6A and 6B are schematic diagrams illustrating a stimulating assembly configured to cause electroporation of nerve cells in accordance with embodiments presented herein;

FIG. 7 is a cross-sectional view of a portion of a stimulating assembly configured for delivery of optically-sensitive elements into the proximity of a recipient's nerve cells in accordance with embodiments presented herein;

FIG. 8 is a cross-sectional view of a portion of another stimulating assembly configured for delivery of optically-sensitive elements into the proximity of a recipient's nerve cells in accordance with embodiments presented herein;

FIG. 9 is a cross-sectional view of a portion of another stimulating assembly configured for delivery of optically-sensitive elements into the proximity of a recipient's nerve cells in accordance with embodiments presented herein;

FIG. 10 is a cross-sectional view of a portion of another stimulating assembly configured for delivery of optically-sensitive elements into the proximity of a recipient's nerve cells in accordance with embodiments presented herein; and

FIG. 11 is a schematic diagram of an auditory brain stimulator configured to develop optically active nerve cells in accordance with embodiments presented herein.

### DETAILED DESCRIPTION

Embodiments presented herein are generally directed to techniques for developing optically active cells (e.g., nerve cells) in a recipient with a stimulating prosthesis (i.e., a device configured to electrically and/or optically stimulate cells of a recipient). An optically active cell is a cell of the recipient that behaves or responds differently to electromagnetic radiation (i.e., light) than the same type of natural cells. As used herein, the “development” of optically active cells refers to the transformation (conversion) of a naturally occurring cell into an optically active nerve through exposure to optically-sensitive elements in combination within a mechanism that enables the optically-sensitive elements to enter the cells. In certain embodiments, an electrical field is applied by a stimulating prosthesis that comprises an auditory prosthesis (e.g., cochlear implant, auditory brainstem stimulator, etc.) to cause electroporation of the nerve cells, thereby enabling the optically-sensitive elements to enter the cells. In other embodiments, focused electromagnetic radiation (e.g., applied by lasers) is applied by an auditory prosthesis to temporarily open pores in a cell, which allow optically-sensitive elements to enter the cells.

For ease of illustration, the techniques for developing optically active cells are primarily described herein with reference to one type of auditory prosthesis, namely a cochlear implant (also commonly referred to as cochlear implant device, cochlear prosthesis, and the like; simply “cochlear implant” herein). However, it is to be appreciated that techniques presented herein for developing optically active cells through the application of an electrical field or focused electromagnetic radiation may be used in conjunction with other stimulating prostheses.

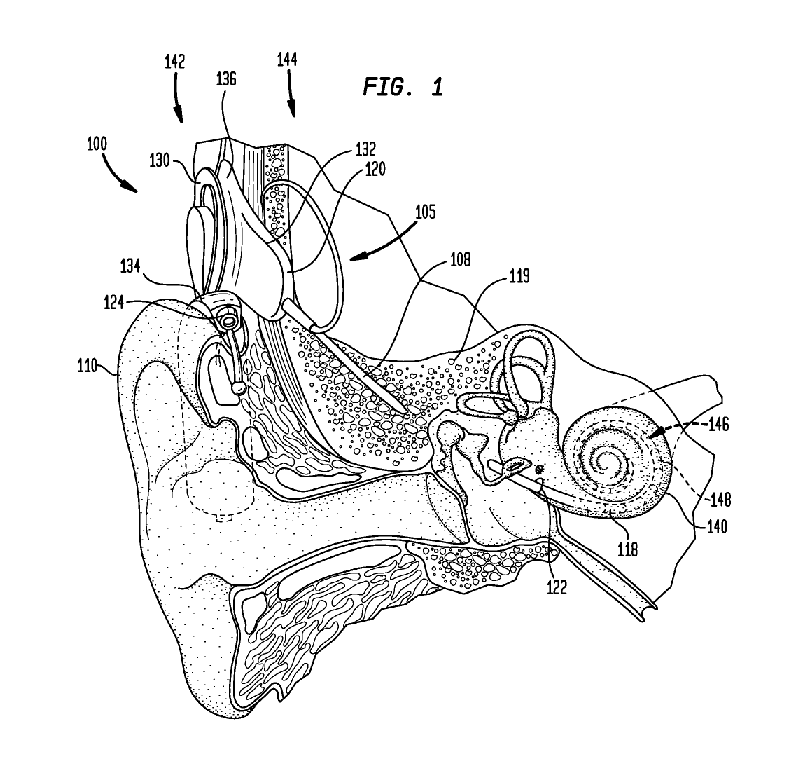

FIG. 1 is perspective view of an exemplary cochlear implant 100 configured to transform a recipient's cochlear nerve cells (e.g., spiral ganglion cells (SGCs)) into optically active (optically sensitive) cells in accordance with embodiments presented herein. The cochlear implant 100 includes an external component 142 and an internal or implantable component 144. The external component 142 is directly or indirectly attached to the body of the recipient and typically comprises one or more sound input elements 124 (e.g., microphones, telecoils, etc.) for detecting sound, a sound processor 126, a power source (not shown), an external coil 130 and, generally, a magnet (not shown) fixed relative to the external coil 130. The sound processor 126 processes electrical signals generated by a sound input element 124 that is positioned, in the depicted embodiment, by auricle 110 of the recipient. The sound processor 126 provides the processed signals to external coil 130 via a cable (not shown).

The implantable component 144 comprises an implant body 105, a lead region 108, and an elongate stimulating assembly 118. The implant body 105 comprises a stimulator unit 120, an internal coil 136, and an internal receiver/transceiver unit 132, sometimes referred to herein as transceiver unit 132. The transceiver unit 132 is connected to the internal coil 136 and, generally, a magnet (not shown) fixed relative to the internal coil 136. Internal transceiver unit 132 and stimulator unit 120 are sometimes collectively referred to herein as a stimulator/transceiver unit 120.

The magnets in the external component 142 and implantable component 144 facilitate the operational alignment of the external coil 130 with the internal coil 136. The operational alignment of the coils enables the internal coil 136 to transmit/receive power and data to/from the external coil 130. More specifically, in certain examples, external coil 130 transmits electrical signals (e.g., power and stimulation data) to internal coil 136 via a radio frequency (RF) link. Internal coil 136 is typically a wire antenna coil comprised of multiple turns of electrically insulated single-strand or multi-strand platinum or gold wire. The electrical insulation of internal coil 136 is provided by a flexible silicone molding. In use, transceiver unit 132 may be positioned in a recess of the temporal bone of the recipient. Various other types of energy transfer, such as infrared (IR), electromagnetic, capacitive and inductive transfer, may be used to transfer the power and/or data from an external device to cochlear implant and FIG. 1 illustrates only one example arrangement.

Elongate stimulating assembly 118 is at least partially implanted in cochlea 140 and includes a contact array 146 comprising a plurality of stimulating contacts 148. Stimulating assembly 118 extends through cochleostomy 122 and has a proximal end connected to stimulator unit 120 via lead region 108 that extends through mastoid bone 119. Lead region 108 couples the stimulating assembly 118 to implant body 105 and, more particularly, stimulator/transceiver unit 120.

In general, cochlear implant 100 stimulates the recipient's auditory nerve cells, bypassing absent or defective hair cells that normally transduce acoustic vibrations into neural activity. In the embodiment of FIG. 1, contact array 146 comprises both optical contacts and electrical contacts that may each be used to stimulate the recipient's cochlea nerve cells to cause a hearing percept (i.e., activate auditory neurons that normally encode differential pitches of sound). As described further below, the electrical contacts may also be used to deliver an electrical field that, in the presence of optically-sensitive elements, enables nerve cells to transform into optically active nerve cells. Alternatively, the optical contacts may be used to deliver focused electromagnetic radiation (e.g., the optical contacts may be laser sources) that, in the presence of optically-sensitive elements, enables nerve cells to transform into optically active nerve cells. As described further below, the cochlear implant 100 is a dual-function device (i.e., a device that is configured to deliver an electrical field and/or electromagnetic radiation to open pores in the cells to enable cell transformation, as well as to stimulate the cells with electromagnetic radiation).

The human auditory system is composed of many structural components, some of which are connected extensively by bundles of nerve cells (neurons). Each nerve cell has a cell membrane which acts as a barrier to prevent intercellular fluid from mixing with extracellular fluid. The intercellular and extracellular fluids have different concentrations of ions, which leads to a difference in charge between the fluids. This difference in charge across the cell membrane is referred to herein as the membrane potential or membrane voltage of the nerve cell. Nerve cells use membrane potentials to transmit signals between different parts of the auditory system.

In nerve cells that are at rest (i.e., not transmitting a nerve signal), the membrane potential at that time is referred to as the resting potential of the nerve cell. Upon receipt of a stimulus, the electrical properties of a nerve cell membrane are subjected to abrupt changes, referred to herein as a nerve action potential, or simply action potential. The action potential represents the transient depolarization and repolarization of the nerve cell membrane. The action potential causes the transmission of a signal along the conductive core (axon) of a nerve cell. Signals may be then transmitted along a group of nerve cells via such propagating action potentials.

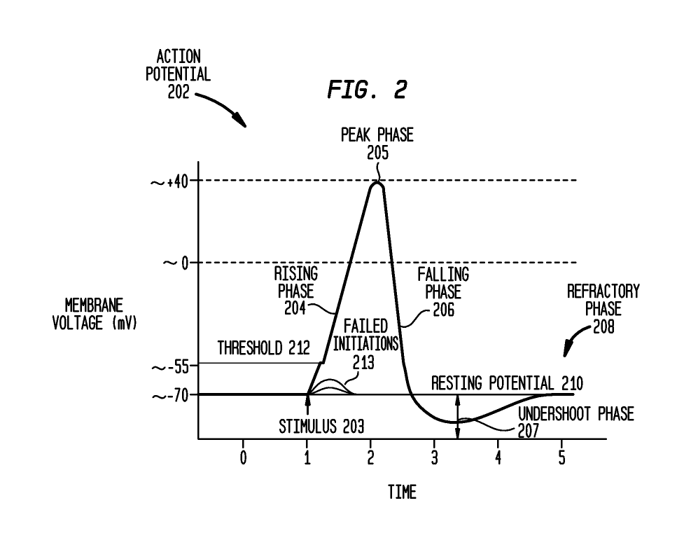

FIG. 2 is a graph illustrating the various phases of an idealized action potential 202 as the potential passes through a nerve cell in accordance with embodiments of the present invention. The action potential is presented as membrane voltage in millivolts (mV) versus time. It is to be appreciated that the membrane voltages and times shown in FIG. 2 are provided for illustration purposes only. The actually voltages may vary and this illustrative example should not be construed as limiting embodiments presented herein.

In the example of FIG. 2, prior to application of a stimulus 203 to the nerve cell, the nerve cell has a resting potential 210 of approximately −70 mV. Stimulus 218 is applied at a first time (1 millisecond (ms) of the graph). In normal hearing, this stimulus is provided by movement of the hair cells of the cochlea. Movement of these hair cells results in the release of a nerve impulse, sometimes referred to as neurotransmitter.

As shown in FIG. 2, following application of stimulus 203, the nerve cell begins to depolarize. Depolarization of the nerve cell refers to the fact that the voltage of the nerve cell becomes more positive following stimulus 203. When the membrane of the nerve cell becomes depolarized beyond the cell's critical threshold 212, the nerve cell undergoes an action potential. This action potential is sometimes referred to as the “firing” of the nerve cell. As used herein, the critical threshold of a nerve cell, group of nerve cells, etc. refers to the threshold level at which the nerve cell, group of nerve cells, etc. will undergo an action potential. In the example illustrated in FIG. 2, the critical threshold 212 for firing of the nerve cell is approximately −50 mV. As would be appreciated, the critical threshold and other transitions may be different for various recipients. As such, the values provided in FIG. 2 are merely illustrative.

The course of this action potential in the nerve cell can be generally divided into five phases. These five phases are shown in FIG. 2 as a rising phase 204, a peak phase 205, a falling phase 206, an undershoot phase 207, and finally a refractory phase 208. During the rising phase 204, the membrane voltage continues to depolarize until the membrane voltage reaches peak phase 205. In the illustrative embodiment of FIG. 2, at this peak phase 205, the membrane voltage reaches a maximum value of approximately 40 mV.

Following peak phase 205, the action potential undergoes the falling phase 206. During the falling phase 206, the membrane voltage becomes increasingly more negative, which is sometimes referred to as hyperpolarization of the nerve cell. This hyperpolarization causes the membrane voltage to temporarily become more negatively charged then when the nerve cell is at rest. This phase is referred to as the undershoot phase 207 of action potential 202. Following the undershoot phase 207, there is a time period during which it is impossible or difficult for the nerve cells to fire. This time period is referred to as the refractory phase 208.

An action potential, such as action potential 202 illustrated in FIG. 2, may travel along a recipient's auditory nerve without diminishing or fading out because the action potential is regenerated each nerve cell. This regeneration occurs because an action potential at one nerve cell raises the voltage at adjacent nerve cells. This induced rise in voltage depolarizes adjacent nerve cells thereby provoking a new action potential therein.

As noted above, the nerve cell must reach a membrane voltage above a critical threshold 212 before the nerve cell may fire. Illustrated in FIG. 2 are several failed initiations 213 which occur as a result of stimuli which were insufficient to raise the membrane voltage above the critical threshold value to result in an action potential.

In traditional hearing, sound pressure waves travel down an individual's auditory pathway (i.e., outer ear, ear canal, and middle ear) until they reach the perilymph (fluid) in the cochlea. As this fluid vibrates in response to the sound energy, the hair cells in the cochlea are displaced to cause depolarization as shown in FIG. 2, thereby triggering an action potential in the cochlear nerve cells. This action potential propagates up the auditory nerve to the auditory centers of the brain.

Due to any of a number of different factors (e.g., infection, loud sounds, drug side effects, etc.), individuals lose the function of the hair cells within the cochlea. In such circumstances, the hair cells cannot depolarize and hence cannot trigger action potentials to send to the auditory nerve. This results in an individual's inability to hear sounds. Cochlear implants operate by emulating the function of the nonfunctional hair cells. More specifically, in response to a received sound, the cochlear implant will generate stimulation pulses targeted to specific cochlea nerve cells to active those nerve cells (i.e., depolarize the nerve cells to generate an action potential).

Present commercial devices offered by the industry use electrical current to stimulate a recipient's nerve cells. In general, the electrical current is delivered within an intensity that is efficient at depolarizing the nerve cells. That is, the electrical current is delivered in a manner that quickly raises the membrane voltages of the target nerve cells above their critical thresholds so as to generate action potentials.

With optical stimulation, pulses of electromagnetic radiation (light) are delivered to the cochlea nerve cells. The electromagnetic radiation is not limited to the portion of the electromagnetic spectrum that is visible to the human eye, commonly referred to as the optical or visible spectrum. Rather, the electromagnetic radiation may comprise other portions of the electromagnetic spectrum such as the ultraviolet, visible, infrared, far infrared or deep infrared radiation.

Irradiating cochlear nerve cells with electromagnetic radiation can induce an action potential which then travels up the auditory nerve, providing the sensation of hearing. Since research into optical stimulation is ongoing, the exact mechanism by which the electromagnetic radiation actives nerve cells is not well understood. However, it is believed that the cochlea nerve cells respond, at least in part, to the heat generated by the electromagnetic radiation. In general, the amount of energy used to stimulate cochlea nerve cells with electromagnetic radiation is high and, as such, the power requirements for the cochlear implant are higher than required in implants that use only electrical stimulation. Also, the response time of cochlea nerve cells to the electromagnetic radiation is reasonably slow due to the fact that the system must wait for the heat energy to dissipate. The slow reaction of the nerve cells may lead to less effective hearing perception, inability to use high rates of stimulation, etc.

Presented herein are techniques that improve responsiveness of nerve cells to optical stimulation. In general, the techniques transform the cochlear nerve cells into active nerve cells. More specifically, the techniques presented herein install optically-sensitive elements (e.g., proteins) which respond to electromagnetic radiation into the walls of the cochlea nerve cells and/or nerve cells in the surrounding areas (e.g., facial nerve). As described further below, the optically-sensitive elements may cause the nerve cells to fire or activate (i.e., generate an action potential) in the presence of electromagnetic radiation, or may prevent the nerve cells from firing or activating in the presence of electromagnetic radiation.

The production of proteins by a cell is coded in the cell's Deoxyribonucleic acid (DNA). Therefore, to install optically-sensitive elements into the nerve cells, the DNA to code them needs to be added to the nerve cell's DNA. In accordance with certain embodiments presented herein, this is done through transfection caused by electroporation (i.e., in response to an applied electric field). It is to be appreciated that other types of transfection (e.g., chemical, viral, etc.) may be used in certain embodiments presented herein. Alternatively, focused electromagnetic radiation may be applied to temporarily open pores in cells. Merely for ease of illustration, embodiments will be primarily described below with reference to electroporation induced transfection.

As used herein, cell transfection refers to techniques by which proteins or other biological materials (e.g., optically-sensitive elements, electrically-sensitive materials, etc.) are introduced into a recipient's cells. Electroporation refers to the application of an electrical field to a cell such that pores are opened in the cell membrane. As the potential difference is applied to the cell, the electrically opened pores in the cell membrane allow material to flow into the cell.

In accordance with certain embodiments presented herein, the electrically opened pores enable optically-sensitive elements (e.g., proteins) to be transfected into the cells. In this way, as described further below, the transfected cells may become optically sensitive. It is to be appreciated that other biological materials may be transfected into a recipient's cells. For example, electrically-sensitive materials (e.g., proteins) may be transfected into the cells to make the cells electrically sensitive. These electrically-sensitive materials may be useful to, for example, to lower the stimulation threshold and make the cell more sensitive to electrical stimulation. Merely for ease of reference, embodiments will be primarily described with reference to the introduction of optically-sensitive elements into a recipient's cells.

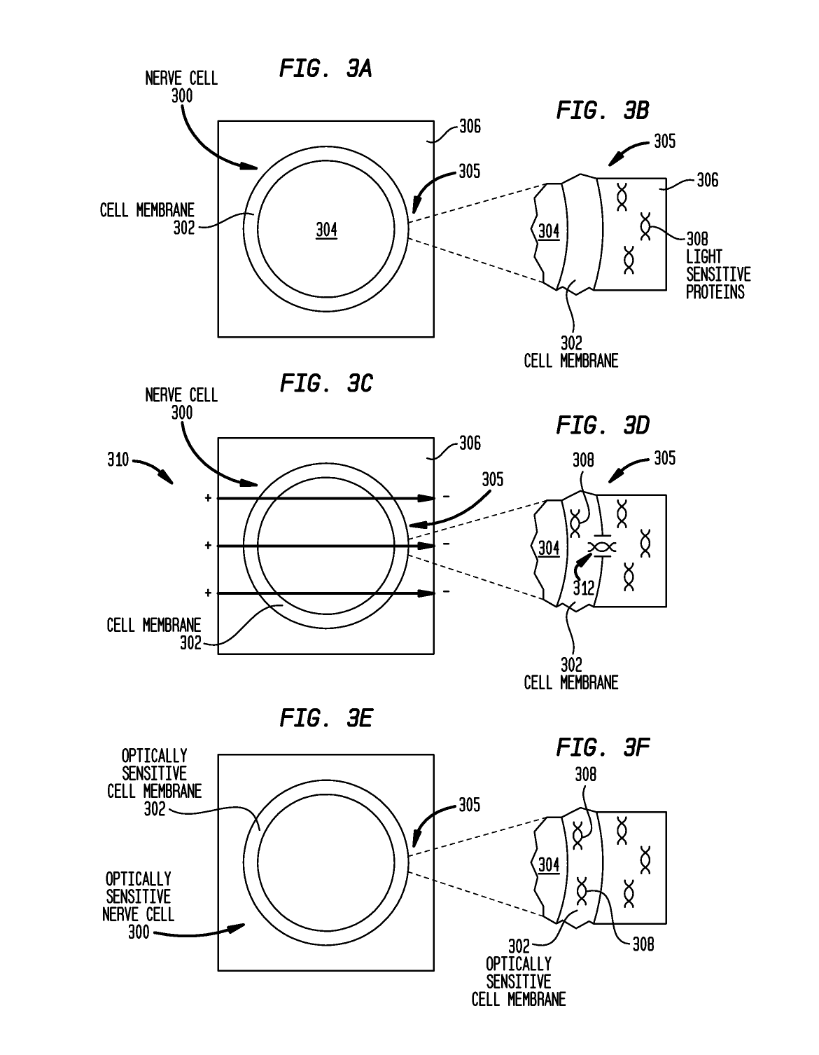

FIGS. 3A, 3C, and 3E are schematic diagrams illustrating a nerve cell prior to, during, and after transformation into an optically-sensitive nerve cell in accordance with embodiments presented herein via electroporation. FIGS. 3B, 3D and 3F are enlarged views of a portion of the nerve cell shown in FIGS. 3A, 3C, and 3E.

More specifically, FIG. 3A is a schematic diagram of a natural nerve cell 300 at rest prior to transformation into an optically-sensitive nerve cell. As shown, the nerve cell 300 comprises a cell membrane 302 that separates the interior 304 of the nerve cell 300 from the surrounding area 306. Generally, the area 306 is a fluid filled space. FIG. 3B is an enlarged view of a portion 305 of nerve cell 300, including a portion of the cell interior 304, a portion of the cell membrane 302, and the area 306 adjacent to the portion 305 of the cell membrane.

As shown in FIG. 3B, optically-sensitive elements 308 are disposed in the area 306 around the nerve cell 300. As described further below, a number of different delivery mechanisms may be used to introduce the optically-sensitive elements 308 into the area 306 around the nerve cell 300.

As shown in the schematic diagram of FIG. 3C, once optically-sensitive elements 308 are introduced into the proximity of the nerve cell 300, an electrical potential (i.e., voltage difference) 310 is applied across the nerve cell 300 in a manner that causes electroporation of the nerve cell. More specifically, as shown in FIG. 3D, the electrical potential across the nerve cell 300 opens up pores 312 in the cell membrane 302 that allow the optically-sensitive elements 308 to enter the cell membrane 302 (i.e., the electroporation causes transfection of the optically-sensitive elements 308 into the nerve cell 300). As described further below, a cochlear implant or other electrically stimulating auditory prosthesis is used to cause electroporation of a nerve cell, such as nerve cell 300.

As shown in FIGS. 3E and 3F, after the electrical potential 310 is removed, the pores 312 in the cell membrane 302 close such that the optically-sensitive elements 308 remain in the cell membrane 302. A cell membrane into which optically-sensitive elements have been introduced is referred to herein as an optically active or optically sensitive cell membrane. Similarly, a nerve cell that includes an optically active cell membrane is referred to herein as an optically active or optically sensitive nerve cell. In general, once the optically-sensitive elements have been transfected into the nerve cells, the division of such optically active nerve cells via natural processes will result in additional optically active nerve cells (i.e., new nerve cells with the optically-sensitive elements embedded in the cell membranes).

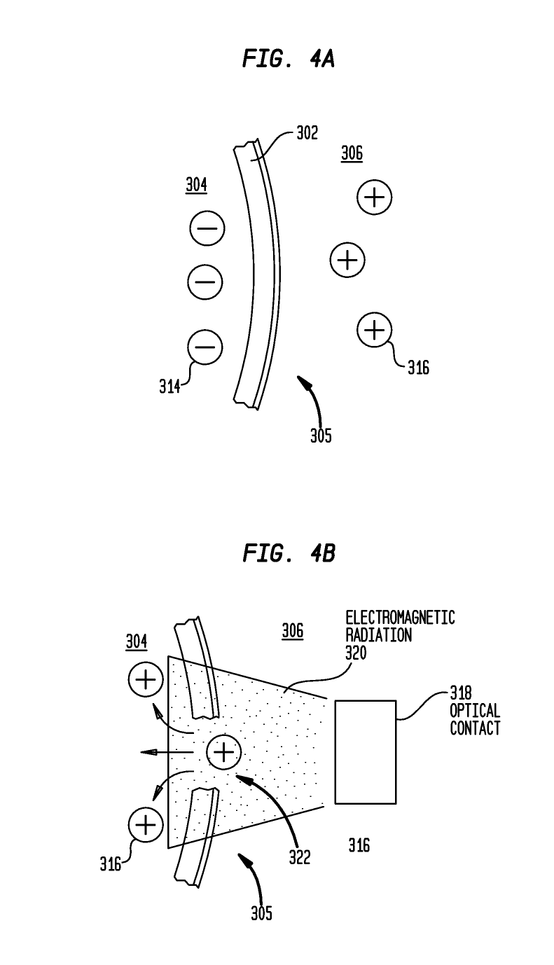

FIG. 4A is a schematic diagram illustrating the portion 305 of optically active nerve cell 300 shown in FIG. 3F prior to optical stimulation. FIG. 4B is a schematic diagram illustrating the portion 305 during optical stimulation.

As explained above, an electrical potential (i.e., a voltage difference) exists across a cochlea nerve cell membrane. The electrical potential across optically sensitive cell membrane 302 is illustrated in FIG. 4A by negatively charged ions 314 within the cell interior 304 and positively charged ions 316 within the area 306. It is to be appreciated that the use of the term “positively” with reference to the ions 316 refers to the charge of the ions 316 relative to the negatively charged ions 314. In other words, the ions 316 have a charge that is more positive than the ions 316, but the charge of the ions 316 may still be negative.

As shown in FIG. 4B, when an optical contact 318 delivers electromagnetic radiation 320 to the optically active cell membrane 302, the optically-sensitive elements 308 (not shown in FIGS. 4A and 4B) disposed in the cell membrane will open up ion channels 322 through the cell membrane. When the ion channels 322 are opened, the positively charged ions 316 enter the cell interior 304, thereby causing changes to the electrical potential across the optically sensitive cell membrane 302. In other words, the optically sensitive cell membrane 302 is depolarized (as described above) so as to generate an action potential.

Due to the presence of the optically-sensitive elements that open up the ion channels 322, the nerve cells 300 of FIG. 4B may activate faster and with less electromagnetic radiation than a natural nerve cell. That is, in accordance with embodiments presented herein, optically active nerve cells may fire without the need to wait for sufficient heat build up from an optical contact.

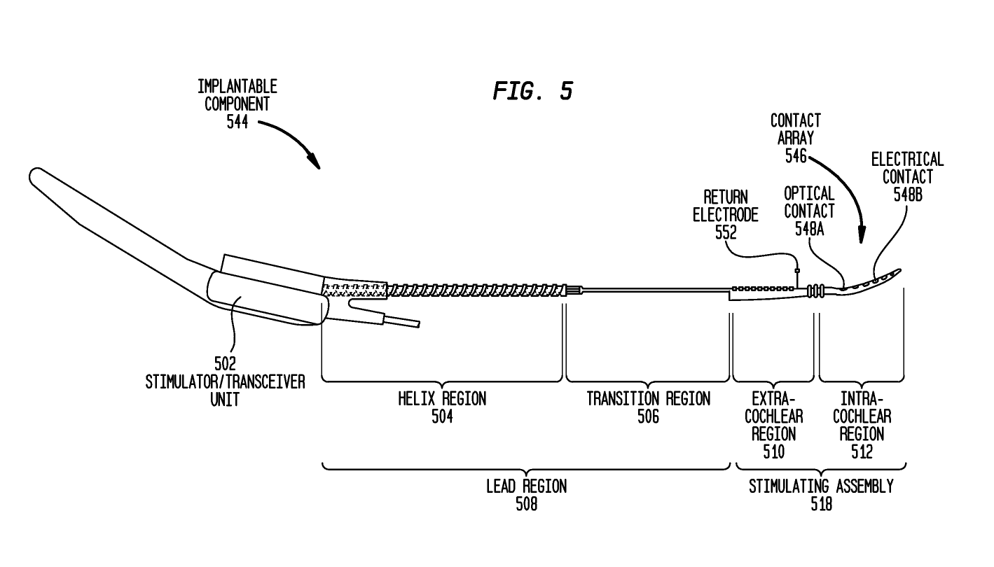

As noted above, electroporation of cochlea nerve cells in accordance with embodiments presented herein is carried out using the electrical stimulating functions of an auditory prosthesis, such as a cochlear implant. FIG. 5 is a simplified side view of an embodiment of implantable component 144 (FIG. 1), referred to herein as implantable component 544. As shown in FIG. 5, implantable component 544 comprises a stimulator/transceiver unit 502 which, as described above, receives encoded signals from an external component of the cochlear implant. Implantable component 544 terminates in a stimulating assembly 518 that comprises an extra-cochlear region 510 and an intra-cochlear region 512. Intra-cochlear region 512 is configured to be implanted in the recipient's cochlea and has disposed thereon an array 546 of contacts. In the illustrative embodiment of FIG. 5, contact array 546 comprises optical contacts 548A and electrical contacts 548B. Contact array 546 may comprise any number of optical or electrical contacts in a variety of arrangements.

Internal component 544 further comprises a lead region 508 coupling stimulator/receiver unit 502 to stimulating assembly 518. Lead region 508 comprises a helix region 504 and a transition region 506. Helix region 504 is a section of lead region 508 in which electrode leads are would helically. Transition region 506 connects helix region 504 to stimulating assembly 518. As described below, optical and/or electrical stimulation signals generated by stimulator/transceiver unit 502 are delivered to contact array 546 via lead region 508. Helix region 504 prevents lead region 508 and its connection to stimulator/transceiver 502 and stimulating assembly 518 from being damaged due to movement of internal component 544 which may occur, for example, during mastication.

As shown in FIG. 5, the internal component 544 also comprises a reference or return electrode 552. The return electrode 552 is configured to be positioned outside a recipient's cochlea and provides a return path in certain electroporation stimulation strategies.

In the embodiments illustrated in FIG. 5, the stimulator/transceiver unit 502 comprises an electromagnetic radiation (EMR) generator (not shown in FIG. 5) that generates optical stimulation signals for delivery to the recipient via optical contacts 548A of stimulating assembly 418. The stimulator/transceiver unit 502 also comprises an electrical stimulation generator (not shown in FIG. 5) configured to generate electrical stimulation signals for delivery to the recipient via electrical contacts 548B. The stimulator/transceiver unit 502 may, in certain examples, include dedicated circuitry that is configured to generate electrical stimulation that causes electroporation of a recipient's nerve cells. In other embodiments, the electrical stimulation signals for electroporation are generated by the same circuitry used for generation of electrical stimulation signals to cause a hearing percept.

As noted, in accordance with embodiments presented herein, electroporation of the cochlea nerve cells is caused by electrical stimulation delivered by an electrically stimulating auditory prosthesis, such as a cochlear implant. An auditory prosthesis may use a number of different electrical stimulation strategies or modes including, for example, using monopolar stimulation, bipolar stimulation, common ground, etc. to cause electroporation of the cochlea nerve cells. The various stimulation modes differ from another in the shape the electrical field generated within each stimulation mode.

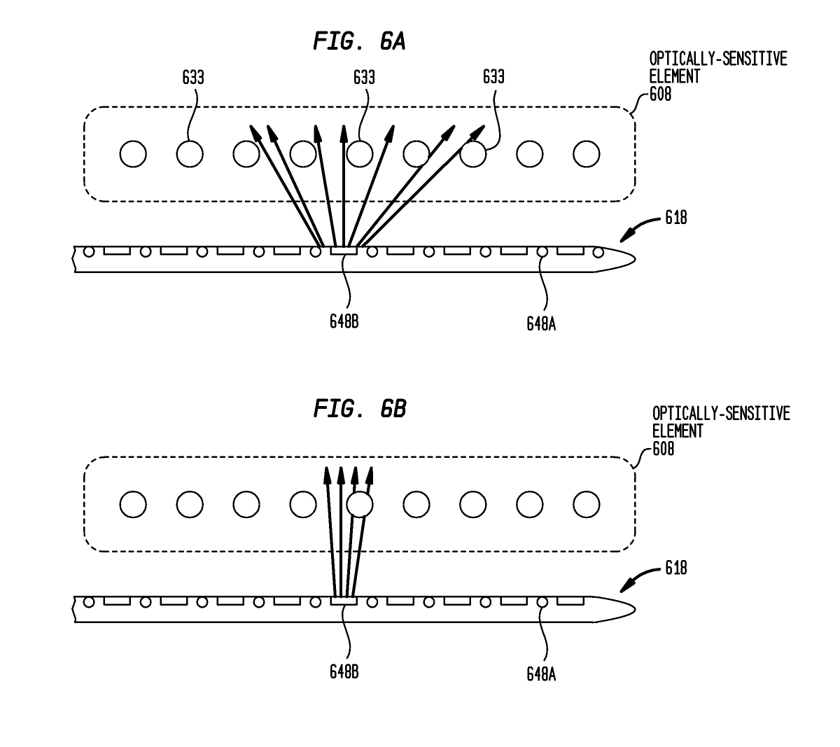

FIGS. 6A and 6B are schematic diagrams each illustrating a portion of a cochlear implant stimulating assembly 618 configured for electroporation of cochlea nerve cells 633. The stimulating assembly 618 comprises a plurality of optical contacts 648A and a plurality of electrical contacts 648B. In the embodiments of FIGS. 6A and 6B, the stimulating assembly 618 is disposed in a recipient's cochlea. Additionally, optically-sensitive elements are positioned in proximity of the nerve cells 633. The optically-sensitive elements are illustrated in FIGS. 6A and 6B by dashed box 608.

In the embodiment of FIG. 6A, one or more electrical contacts 648B operate in accordance with a stimulation mode that results in the generation of a wide electrical field 655 that causes electroporation of a substantially large population of nerve cells 633. The wide electrical field 655 may be generated, for example, using a bipolar stimulation mode, a common ground stimulation mode, a monopolar stimulation mode, etc. In general, the electrical field causes electroporation of the nerve cells 633 within the electric field 655 such that the optically-sensitive elements 608 are transfected into the nerve cells.

In certain circumstances, it may be desirable to limit the area of nerve cells 633 that are subject to electroporation. For example, it may be useful to cause electroporation (and thus transfect) nerve cells 633 in a small part of the cochlea having non-functional hair cells without affecting other parts of the cochlea. To this end, FIG. 6B illustrates the use of a stimulation mode where a combination of electrical contacts 648B operate in accordance with a stimulation mode that generates a narrow or focused electrical field 657. The focused electrical field 657 may be generated, for example, using multipolar stimulation (sometimes referred to as phased array stimulation). In the embodiment of FIG. 6B, a smaller area of nerve cells 633 will be stimulated than the other stimulation modes of FIG. 6A (i.e., stimulation modes that generate a wide electrical field 655).

In accordance with certain embodiments, multipolar or other focused stimulation modes may be used to “steer” the electric field to other regions of the cochlea and/or the surrounding area so that transfection can occur at other sites. This may be useful in treating facial nerve stimulation, as discussed further below. Additionally, some types of cells can be damaged by repetitive stimulation. Multipolar or other focused stimulation modes could be used to steer the electroporation electric field away from these types of cells.

In accordance with certain electroporation techniques presented herein, a collection of stimulating contacts 648B may operate as a single electrical contact to deliver electroporation stimulation signals. In other embodiments, individual electrical contacts may operate separately to deliver electroporation stimulation signal. In certain embodiments, electroporation may be performed by simultaneously polarizing one or more stimulating contacts 648 relative to a reference electrode (not shown in FIGS. 6A and 6B) located outside of the recipient's cochlea. In another embodiment electroporation utilizes bimodal current delivery mode.

In accordance with embodiments presented herein, the electroporation stimulus is typically provided in a square wave configuration where pulses have a pulse width in the range of approximately 100 microseconds (μs) to approximately 500 ms. In certain embodiments, the pulses have a pulse width of approximately 30 ms. Multiple pulses in the range of approximately 25 to 200 pulses, and more typically from 50 to 100 pulses, may be used. In certain embodiments, ramped waveforms may also be used that may provide a faster onset of electroporation dielectric breakdown of the cell membrane while minimizing current delivery.

As noted above, in accordance with embodiments presented herein, a cochlear implant or other auditory prosthesis is configured to cause electroporation of a recipient's nerve cells in order to make those cells optically active (i.e., sensitive to light). In order for the electroporation to make nerve cells optically active, optically-sensitive elements are first introduced into the proximity of the nerve cells.

In general, there are different families of optically-sensitive elements which can be used to transform cells into optically active cells. Certain optically-sensitive elements may be used to make cells fire in the presence of electromagnetic radiation. For example, Channelrhodopsin-1 (ChR1), Channelrhodopsin-2 (ChR2), Volvox channelrhodopsin-1 (VChR1), VChR1-ChR2 hybrids, and Channelrhodopsin-halorhodopsin gene fusions (ChR2-2A-Halo) may be use to make cells activate in the presence of electromagnetic radiation. In particular, ChR2 may make cells activate in the presence of blue light, while VChR1 may make cells activate in the presence of yellow light.

Certain other optically-sensitive elements may be used to inhibit the ability of cells to fire in the presence of electromagnetic radiation. For example, halorhodopsin (NpHR), enhanced halorhodopsins (eNpHR2.0 and eNpHR3.0), archaerhodopsin (Arch), Archaerhodopsin from Halorubrum (ArchT), Halorhodopsin from N. pharaonis (Halo/NpHR), Leptosphaeria maculans fungal opsins (Mac), and enhanced bacteriorhodopsin (eBR) may be employed to inhibit cell firing. In particular, NpHR may be used to inhibit cell firing in the presence of yellow light, while Arch may be used to inhibit cell firing in the presence of yellow/green light.

It is to be appreciated that a number of different optically-sensitive elements may be used in the embodiments presented herein to transform cells into optically active cells. As such, the optically-sensitive elements identified herein are merely illustrative and any optically-sensitive elements now known or later developed may be used in alternative embodiments.

As described below, optically-sensitive elements may be introduced into the proximity of a recipient's nerve cells using a number of different delivery mechanisms. The optically-sensitive elements may be delivered to the cochlea at the time the stimulating assembly is inserted into the cochlea, or shortly before or after insertion of the stimulating assembly. The optically-sensitive elements may be provided in a diffusible form, such as incorporated in or associated with a biodegradable or biocompatible viscous liquid or gel solution surrounding the stimulating assembly of the cochlear implant. The viscous liquid or gel solution may comprise polyacrylic acid (PAA), polyvinyl alcohol (PVA), polylactic acid (PLA) and/or polyglycolic acid (PGA). In certain examples, pluronic F127 (BASF) at less than approximately 30% solution may also be used to stabilize the media containing the optically-sensitive elements.

In certain embodiments, optically-sensitive elements may be presented in a diluent solution which comprises ions and optically-sensitive elements or diluted in sterile distilled deionized water, provided that the diluent solution does not substantially adversely affect the efficiency of electroporation. Water may be used as the diluent, with the nucleic acid molecule provided typically at a concentration of from approximately ten (10) nanograms (ngs) per microliter (μl) to approximately 1 microgram (μg) per μl. The nucleic acid molecules may be provided within a buffered solution, such as phosphate-buffered saline, or a perilymph-like solution, and may be in the presence of divalent cation chelators such as ethylenediaminetetraacetic acid (EDTA) for stabilization of the molecules.

In certain examples, a delivery apparatus that is separate from the cochlear implant is used to deliver the optically-sensitive elements into the proximity of the nerve cells prior to, during, or after implantation of the auditory prosthesis. For example, the optically-sensitive elements are delivered to the cochlea using a catheter or cannula which is not associated with the cochlear implant. In these embodiments, the catheter or cannula may be inserted into the appropriate chamber(s) of the cochlea and a solution comprising the optically-sensitive elements is delivered. The catheter or cannula is then withdrawn, the cochlear implant is introduced and electroporation takes place, as described elsewhere herein.

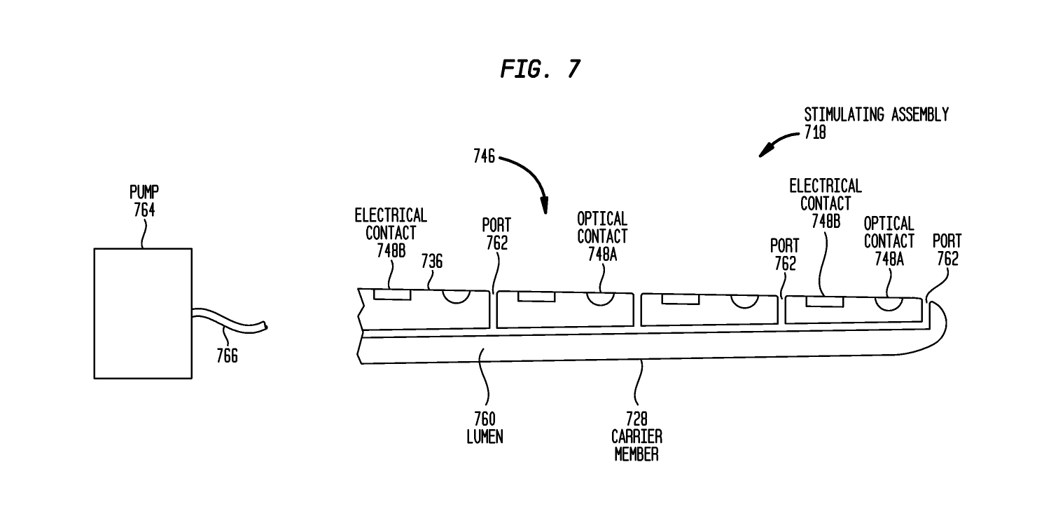

In accordance with certain embodiments presented herein, a cochlear implant is configured to deliver the optically-sensitive elements and to cause electroporation of a recipient's nerve cells. A cochlear implant in accordance with embodiments presented may include a number of different delivery mechanisms that introduce optically-sensitive elements into the proximity of cochlea nerve cells. For example, FIG. 7 is a cross-sectional view of a portion of a stimulating assembly 718 configured for delivery of optically-sensitive elements into the proximity of a recipient's nerve cells via an internal lumen.

More specifically, the stimulating assembly 718 comprises a carrier member 728 configured to be implanted into a recipient's cochlea. Disposed in the carrier member 728 is an array 746 of stimulating contacts. The contact array 746 comprises a plurality of optical contacts 748A and a plurality of electrical contacts 748B disposed along a first surface 736 of the carrier member 728. As shown, the stimulating assembly 718 also comprises an elongate lumen 760 that extends there through. The lumen 760 is connected to the first surface of the carrier member 728 via a plurality of ports 762. A proximal end of the lumen 760 is configured to be connected to a pump 764 via a catheter or tube 766. The pump 764 may be, for example, a mechanical infusion pump, an osmotic pump, etc.

In the embodiment of FIG. 7, optically-sensitive elements are initially disposed in the pump 764 or in a reservoir (not shown) fluidically coupled to the pump and the stimulating assembly 718 is implanted in the recipient's cochlea. The optically-sensitive elements are disposed in a fluid or other solution that is forced from the pump 764 through the catheter 766 into the lumen 760. The fluid may then exit the lumen 760 via the ports 762. When the fluid exits the ports 762, the optically-sensitive elements within the fluid may be in proximity to the cochlea nerve cells. Subsequently, the electrical contacts 748B may cause electroporation of the cochlea nerve cells as described above.

FIG. 7 illustrates a specific implementation where a pump 764 is used to introduce the fluid that includes the optically-sensitive elements into the lumen 760. In an alternative embodiment, the fluid that includes the optically-sensitive elements may be introduced into the lumen 760 prior to implantation and the lumen may then be substantially sealed. In certain such embodiments, the ports 762 may be omitted and the carrier member 728 may be formed from a material than enables the fluid to elute from the carrier member into the cochlea following implantation. Alternatively, the ports 762 may be present, but sealed during implantation. The seals may be removed or may dissolve during or after implantation to enable the fluid to exit via the ports.

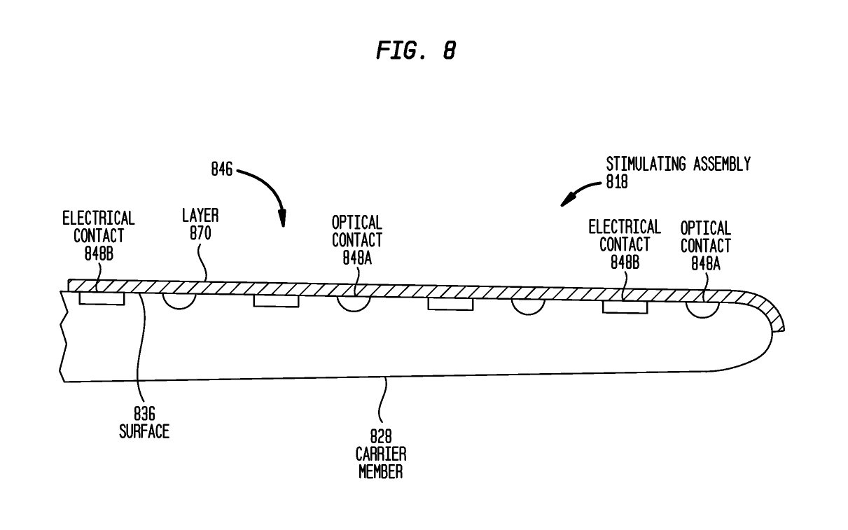

FIG. 8 is a cross-sectional view of a portion of another stimulating assembly 818 configured for delivery of optically-sensitive elements into the proximity of a recipient's nerve cells. The stimulating assembly 818 comprises a carrier member 828 configured to be implanted into a recipient's cochlea. Disposed in the carrier member 828 is an array 846 of stimulating contacts. The contact array 846 comprises a plurality of optical contacts 848A and a plurality of electrical contacts 848B disposed along a first surface 836 of the carrier member 828.

In the embodiment of FIG. 8, a layer or coating 870 of optically-sensitive elements is disposed on the surface 836 of the carrier member 828. More particularly, the optically-sensitive element layer 870 is, prior to implantation, in a solid or gel form at the surface 836. After implantation, moisture within the cochlea causes the optically-sensitive element layer 870 to turn to a liquid form and/or to be resorbed so as to release the optically-sensitive elements within the layer 870.

As an alternative to the layer 870 shown in FIG. 8, a delivery accessory may be disposed on the surface 836 of the carrier member 828. In such embodiments, the delivery accessory may be in the form of a sleeve (e.g., collar, ring, band, or the like) or sheath configured to partially or completely wrap around part of the carrier member 828. The delivery accessory may be a solid that includes optically-sensitive elements. In such embodiments, after implantation, the delivery accessory 870 may elute the optically-sensitive elements or may be resorbed so as to release the optically-sensitive elements within the accessory.

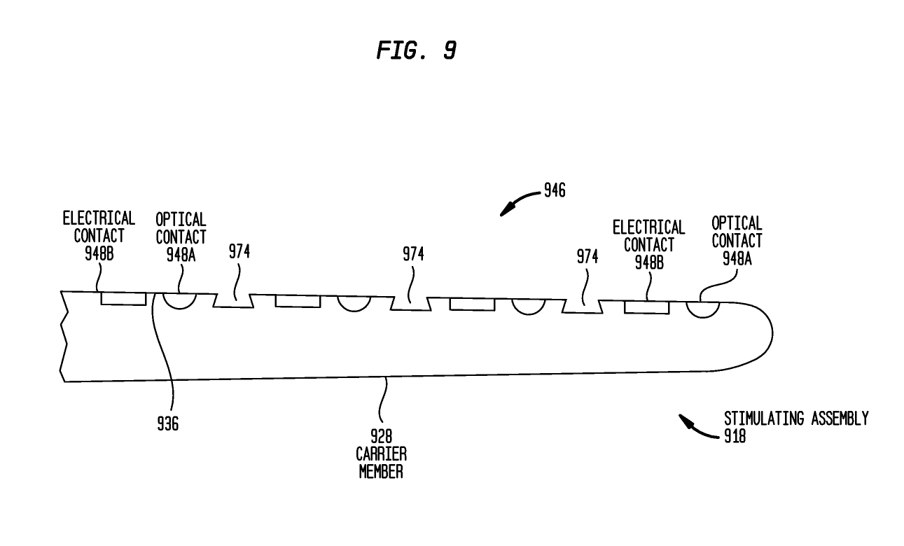

FIG. 9 is a cross-sectional view of a portion of another stimulating assembly 918 configured for delivery of optically-sensitive elements into the proximity of a recipient's nerve cells. The stimulating assembly 918 comprises a carrier member 928 configured to be implanted into a recipient's cochlea. Disposed in the carrier member 928 is an array 946 of stimulating contacts. The contact array 946 comprises a plurality of optical contacts 948A and a plurality of electrical contacts 948B disposed along a first surface 936 of the carrier member 928.

As shown, surface features 974 are disposed in the surface 936 of the carrier member. In the embodiment of FIG. 9, the surface features 974 comprise indents or grooves configured to have optically-sensitive elements positioned therein prior to implantation of the stimulating assembly 918 into the cochlea. In certain such embodiments, the optically-sensitive elements may be disposed in a solid capsule that is positioned within the surface features 974. In such embodiments, the capsule may be configured to be resorbed or to elute the optically-sensitive elements after implantation. Alternatively, the optically-sensitive elements may be disposed in a gel that is positioned within the surface features 974. In such embodiments, the gel may be configured to turn to a liquid form and/or to be resorbed so as to release the optically-sensitive elements.

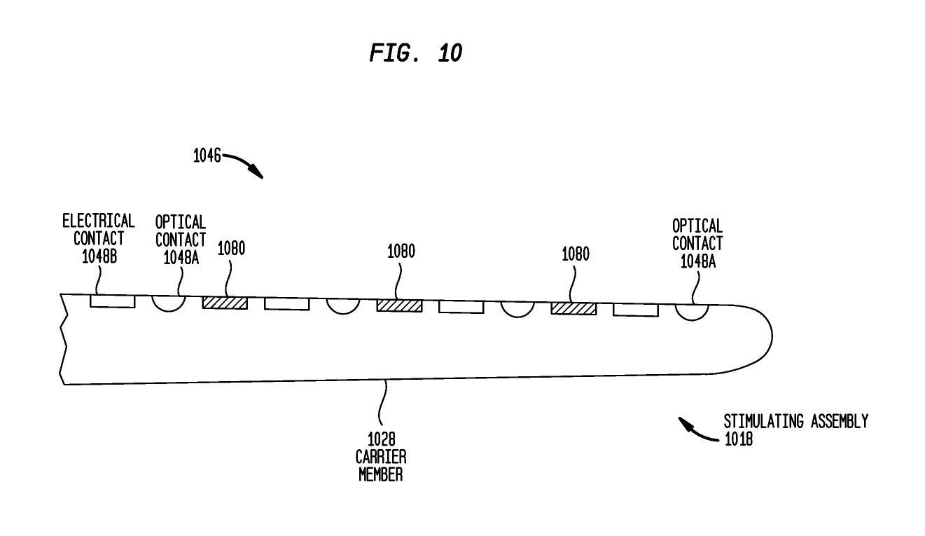

FIG. 10 is a cross-sectional view of a portion of a still other stimulating assembly 1018 configured for delivery of optically-sensitive elements into the proximity of a recipient's nerve cells. The stimulating assembly 1018 comprises a carrier member 1028 configured to be implanted into a recipient's cochlea. Disposed in the carrier member 1028 is an array 1046 of stimulating contacts. The contact array 1046 comprises a plurality of optical contacts 1048A and a plurality of electrical contacts 1048B disposed along a first surface 1036 of the carrier member 1028.

As shown, the carrier member 1028 comprises a plurality of delivery portions 1080 in which optically-sensitive elements are disposed. The delivery portions 1080 are configured to be resorbed or to elute the optically-sensitive elements after implantation.

As noted above, optically-sensitive elements may be introduced into cochlea nerve cells or other nerve cells to make those cells optically active or sensitive. An optically active cell is a cell of the recipient that behaves or responds differently to electromagnetic radiation than the same type of natural cells. In certain embodiments, an optically active nerve cell may be configured to fire or activate (i.e., generate an action potential) in the presence of electromagnetic radiation. Alternatively, an optically active nerve cell may be configured to resist firing or activation in the presence of electromagnetic radiation. As described below, optically active nerve cells may be used in a number of different manners within a cochlear implant or other electrically stimulating auditory prosthesis.

For example, the cochlea is arranged in a tonotopic fashion such that different locations of the cochlea are more sensitive to different wavelengths of sound. In particular, apical regions of the cochlea are more sensitive to longer wavelength sounds, while basal regions of the cochlea are more sensitive to shorter wavelength sounds. Traditional cochlear implants take advantage of this tonotopic organization by assigning different electrical contacts to different wavelengths of sound. That is, when sounds with longer wavelengths (low frequency) are received, the cochlear implant uses the apical electrical contacts to stimulate the cochlea. This stimulates nerve cells in the apical region of the cochlea, giving the recipient the sensation of low frequency sound. Similarly, when sounds with shorter wavelengths (high frequency), the cochlear implant uses the basal electrical contacts, giving the sensation of high frequency sound.

The above tonotopical principals are also applicable to optical stimulation. For example, a collection of light sources may be used to stimulate different regions of the cochlea depending on the frequency of sound received by the sound processor. Low frequency sounds would activate light sources at the apical end of the array, and high frequency sounds would activate light sources at the basal end of the array. This emulates the behavior of the electrical stimulation in traditional Cochlear implants.

It may also be possible to transfect cochlea nerve cells with optically-sensitive elements which are receptive to different wavelengths of light. For example, apical regions of a recipient's cochlea could be transfected with proteins or other elements that respond to longer wavelengths of light (i.e., open up ion channels in the cells in response to longer wavelengths of light), while basal regions could be transfected with proteins or other elements that response to shorter wavelengths of light (i.e., open up ion channels in the cells in response to longer wavelengths of light). It is to be appreciated that this correlation of optical wavelength or regions of the cochlear is merely illustrative, and the wavelength of light need not correspond to the wavelength of received sound in other embodiments.

In such embodiments, a small region of nerve cells could be coded to respond to a particular wavelength of light, while the rest of the cochlea could be inhibited by that same wavelength of light. Furthermore, different regions of the cochlea would be made to respond to different wavelengths of light, and all other regions of the cochlea would be inhibited by the wavelengths that are not meant for those regions. This would allow the use of a single light source which could deliver light to the entire length of the cochlea. Using multipolar stimulation to transfect the nerve cells could ensure the region of stimulation for a particular wavelength of light was reasonably focused on the intended region of the cochlea.

When a recipient's cochlea is transfected in such a tonotopical manner, a cochlear implant could also be configured to deliver different wavelengths of light to the nerve cells. More specifically, when low frequency sounds (long wavelengths) are received, the cochlear implant could deliver a pulse of light with a long wavelength which stimulates only the nerve cells at the apical region of the cochlea. Conversely, when high frequency sounds (short wavelengths) are received, the implant could deliver a pulse of light with a short wavelength which stimulates only the nerve cells at the basal region of the cochlea. Such embodiments may use a number of light sources that each provide a different wavelength of light, or as noted above a single light source which can provide different wavelengths of light (e.g., one light source can be used to flood the cochlea with different wavelengths of light, and only those cells which are sensitive to that frequency of light will be stimulated).

In certain embodiments, optically active nerve cells may be used in cochlear implants that deliver both optical and electrical stimulation signals to a recipient. In particular, the presence of optically active nerve cells may reduce the threshold of optical stimulation needed to fire the nerve cells (relative to optical stimulation of nature nerve cells), thereby reducing the overall threshold of stimulation when optical stimulation is used alone or in combination with electrical stimulation.

Facial nerve stimulation occurs when electrical charge from a cochlear implant activates neurons in the facial nerve. This may result in facial twitching or pain to the recipient. In certain circumstances, facial nerve stimulation may occur when a subset of electrical contacts is used for stimulation. In this case, conventional arrangements may disable the use of those electrical contacts. For some recipients, facial nerve stimulation occurs at high current levels. In those cases, it may be possible to prevent facial nerve stimulation by reducing the threshold and comfort levels of stimulation signals. In both methods, there is the risk that the recipient is not receiving the full benefit of the cochlear implant.

As noted above, an optically active nerve cell may be configured to resist firing or activation in the presence of electromagnetic radiation. That is, the nerve cells can be transfected with optically-sensitive elements that inhibit a nerve cell from firing in the presence of electromagnetic radiation. In such embodiments, rather than opening ion channels through a cell membrane, these optically-sensitive elements prevent ion channels from opening and effectively raise the critical threshold of those nerve cells with respect to electromagnetic radiation. Such optically active nerve cells may be used to prevent facial nerve stimulation.

More specifically, nerve cells within the facial nerve may be altered so that they carry the optically-sensitive elements that inhibit cell firing on in the presence of electromagnetic radiation. In these embodiments, when stimulation (electrical or optical) occurs to evoke a hearing precept, electromagnetic radiation may be delivered to the facial nerve to prevent unwanted facial nerve stimulation. The electromagnetic radiation source may be activated when stimulation occurs on any stimulating contact, or when stimulation occurs on the specific set of stimulating contacts that have been determined to cause facial nerve stimulation.

Tinnitus is a symptom of a number of conditions which causes sufferers to hear sound, typically a ringing, when there is no external source of sound. The mechanisms of tinnitus are not well understood, however most causes seem to be related to hearing loss, with the characteristics of the tinnitus correlating to those of the hearing loss.

Several theories into the mechanisms of tinnitus revolve around the function of the hair cells of the cochlea. Inner hair cells (IHCs) are the hair cells which pick up sound, while outer hair cells (OHCs) act as amplifiers to boost soft sounds. Damage to the cochlea usually affects OHCs more than IHCs. It is theorized that this difference in damage levels may cause the IHCs to provide action potentials without the presence of sound, which is perceived as tinnitus.

Optically active nerve cells may be useful in the treatment of tinnitus. For example, it may be possible to transfect nerve cells associated OHCs with optically-sensitive elements which activate those cells, or to transfect nerve cells associated with IHCs with optically-sensitive elements which inhibit the firing of those cells, when light is shone upon them. An electromagnetic radiation source could be activated at times when no sound is present. That way, the normal action of the cochlear implant is not inhibited. Alternatively, the electromagnetic radiation source could remain switched on permanently, so long as the function of the cochlear implant is not impaired.

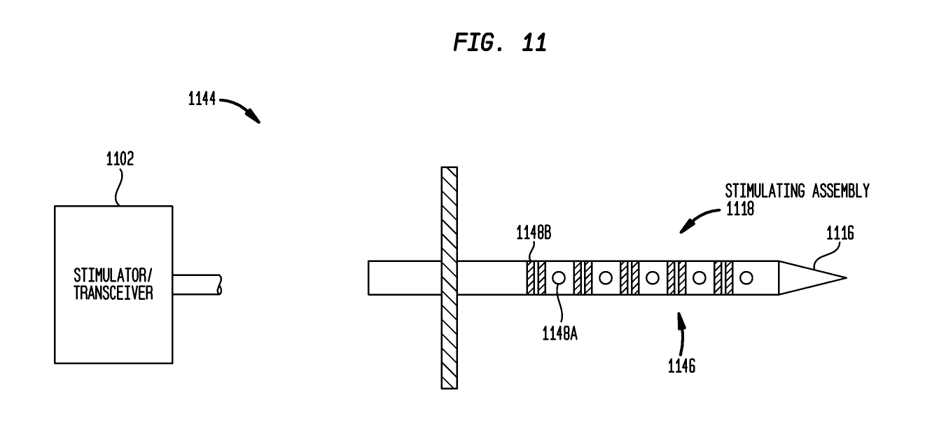

Embodiments have been primarily described with reference to a cochlear implant. However, as noted above, embodiments may be used in alternative electrically stimulating auditory prostheses or other electrically stimulating implantable medical devices. For example, FIG. 11 illustrates an implantable component 1144 of auditory brain stimulator configured to transform natural nerve cells into optically active nerve cells. The implantable component 1144 of FIG. 11 may operate with an external component that is similar to those described above with reference to FIGS. 1 and 5.

Auditory brain stimulators are used to treat a smaller number of recipients, such as those with bilateral degeneration of the auditory nerve. Auditory brain stimulators may also be used to treat disorders such as Parkinson's Disease, Dyskinesia, etc.

In the embodiment of FIG. 11, the implantable component 1144 of the auditory brain stimulator comprises a stimulating assembly 1118 configured to be positioned, for example, proximal to the recipient's brainstem. In certain embodiments, the stimulating assembly 1118 is implanted in the recipient's inferior colliculus. The stimulating assembly 1118 has a distal tip 1116 end that assists in passage of the stimulating assembly 1118 into the brain or portions thereof, such as the inferior colliculus, while causing relatively minimal trauma to the sensitive tissues of the brain. The tip 1116 can be formed of a biocompatible material, such as stainless steel, platinum-iridium alloy or other metals. The tip 1116 may also be formed from a material selected from the group comprising silicone, polytetrafluoroethylene (PTFE), polyurethane, other polymers, and polymer-coated substrates such as silicone-coated platinum and parylene-coated platinum. In certain embodiments, the diameter of the stimulating assembly 1118 can decrease near the tip 1116.

The stimulating assembly 1118 comprises a contact array 1146 of optical contacts 1148A and electrical contacts 1148B. When implanted, the electrical contacts 1148B are configured to apply electrical stimulation signals so as to cause electroporation of adjacent nerve cells. Optically-sensitive elements may be delivered to the site of implantation of the stimulating assembly 1118 via any of the delivery mechanisms described above. In this way, the electroporation of the nerve cells by the electrical contacts 1148B may transform those nerve cells into optically active nerve cells for subsequent use during stimulation by the optical contacts 1148A and/or electrical contacts 1148B.

The above embodiments have primarily been described with reference to the delivery of an electric field to cause electroporation of a recipient's nerve cells. As noted above, in alternative embodiments focused electromagnetic radiation, instead of an electric field, may be delivered to the recipient's nerve cells to opening pores in those cells. To this end, the optical contacts shown in FIGS. 5-11 may be configured to deliver focused electromagnetic radiation (e.g., operate as laser sources) to the nerve cells to open pores that enable the optically-sensitive elements to enter the nerve cells.

As noted above, the techniques presented herein may provide one or more benefits including, for example, enabling efficient use of optical stimulation of a recipient's cells. More specifically, the techniques presented herein may remove the high power requirements and latency associated with conventional optical stimulation. The techniques may also enable the use of focused optical stimulation using an electromagnetic radiation source that illuminates the entire cochlea, as well as the ability to stimulate specific areas of the cochlea using a single electromagnetic radiation source which emits different wavelengths of light.

The techniques presented herein may also enable the use of optical stimulation in manner that reduces the overall electrical stimulation levels. More specifically, by delivering electromagnetic radiation to optically active cochlea nerve cells, those optically active nerve cells can be held in a state which requires less energy to activate them through electrical stimulation.

Furthermore, the techniques presented herein may enable the use of an electromagnetic radiation source to prevent activation of facial nerve cells during stimulation, thus making it easier and quicker for clinicians to fit cochlear implants to recipients. Similarly, the presented techniques may enable cochlear implants to be used to treat tinnitus.

Certain aspects presented herein are embodied as a method that comprises introducing optically-sensitive elements into the proximity of a recipient's cells, and applying an electrical field or electromagnetic radiation to cells of the recipient with a stimulating prosthesis (e.g., auditory prosthesis) such that the optically-sensitive elements are transfected into the cells. The stimulating prosthesis may be an auditory prosthesis comprising a sound processor configured to process sounds, and the method further comprises delivering electromagnetic radiation to stimulate different active cells depending on the frequency of sound processed by the sound processor. As such, the stimulating prosthesis a dual-function device (i.e., a device that is configured to deliver an electrical field and/or electromagnetic radiation to open pores in the cells as well as to stimulate the cells with electromagnetic radiation and/or electrical stimulation).

In certain embodiments, introducing optically-sensitive elements into the proximity of the cells comprises introducing optically-sensitive elements configured to, after transfection, facilitate the firing of the cells in the presence of electromagnetic radiation. In further embodiments, introducing optically-sensitive elements into the proximity of the cells comprises: introducing optically-sensitive elements configured to, after transfection, inhibit the firing of the cells in the presence of electromagnetic radiation. The method may further comprise delivering a focused electrical field to a population of the cells such that the optically-sensitive elements are transfected into the cells and/or delivering a wide electrical field to a population of the cells such that the optically-sensitive elements are transfected into the cells.

In certain embodiments, the stimulating prosthesis is a cochlear implant having a stimulating assembly configured to be implanted into the recipient's cochlea, and the method further comprises: applying the electrical field to nerve cells of the recipient's cochlea with one or more electrical contacts disposed in the stimulating assembly. In further embodiments, the method comprises delivering an electrical field to nerve cells of the recipient's facial nerve with the one or more electrical contacts disposed in the stimulating assembly.

In other embodiments, the stimulating prosthesis is an auditory brainstem implant having a stimulating assembly configured to be implanted into the recipient's brainstem, and the method further comprises applying the electrical field to nerve cells of the recipient's brainstem with one or more electrical stimulating contacts disposed in the stimulating assembly.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

What is claimed is:

1. A stimulating prosthesis, comprising:
a delivery mechanism configured to introduce optically-sensitive elements into a proximity of cells of a recipient; and
a stimulating assembly comprising one or more electrical stimulating contacts configured to apply an electrical field to the cells of the recipient so such that the optically-sensitive elements are transfected into the cells during the electroporation of the cells.

2. The stimulating prosthesis of claim 1, wherein after transfection, the optically-sensitive elements are disposed in cell membranes of the cells and are configured to facilitate initiation of action potentials by the cells in the presence of electromagnetic radiation.

3. The stimulating prosthesis of claim 1, wherein after transfection, the optically-sensitive elements are disposed in cell membranes of the cells and are configured to inhibit initiation of action potentials by the cells in the presence of electromagnetic radiation.

4. The stimulating prosthesis of claim 1, wherein the delivery mechanism is configured to introduce optically-sensitive elements configured to facilitate initiation of action potentials by the optically active nerve cells in the presence of electromagnetic radiation in the proximity of certain cells and introduce optically-sensitive elements configured to inhibit initiation of action potentials by the optically active nerve cells in the presence of electromagnetic radiation in the proximity of other cells.

5. The stimulating prosthesis of claim 1, wherein the one or more electrical stimulating contacts are configured to deliver a focused electrical field to a population of the cells.

6. The stimulating prosthesis of claim 1, wherein the one or more electrical stimulating contacts are configured to deliver a wide electrical field to a population of the cells.

7. The stimulating prosthesis of claim 1, wherein the stimulating assembly is configured to be implanted in a recipient's cochlea such that the one or more electrical stimulating contacts are configured to apply the electrical field to nerve cells of the recipient's cochlea or surrounding area.

8. The stimulating prosthesis of claim 1, wherein the one or more electrical stimulating contacts are configured to apply the electrical field to nerve cells of the recipient's facial nerve.

9. The stimulating prosthesis of claim 1, wherein the one or more electrical stimulating contacts are configured to apply the electrical field to nerve cells within the recipient's brainstem.

10. The stimulating prosthesis of claim 1, further comprising:
one or more optical stimulating contacts configured to optically stimulate the optically active cells with electromagnetic radiation.

11. The stimulating prosthesis of claim 1, further comprising:
a sound processor configured to process sounds; and
a collection of electromagnetic sources configured to deliver electromagnetic radiation to different optically active cells depending on the frequency of sound processed by the sound processor.

12. The stimulating prosthesis of claim 1, wherein the delivery mechanism comprises:
a lumen disposed in the stimulating assembly in which optically-sensitive elements may be positioned; and
a plurality of ports in the stimulating assembly connecting the lumen to an outer surface of the stimulating assembly for introduction of the optically-sensitive elements in the lumen to the proximity of the cells.

13. The stimulating prosthesis of claim 1, wherein the delivery mechanism comprises an outer surface of the stimulating assembly on which the optically-sensitive elements are disposed prior to implantation into the recipient.

14. The stimulating prosthesis of claim 1, wherein the delivery mechanism comprises a portion of the stimulating assembly configured to elute the optically-sensitive elements after implantation into the recipient.

15. The stimulating prosthesis of claim 1, wherein the delivery mechanism comprises one or more surface features formed in the stimulating assembly into which the optically-sensitive elements are positioned prior to implantation into the recipient.

16. A stimulating prosthesis, comprising:
a delivery mechanism configured to introduce optically-sensitive elements into a proximity of a recipient's cells; and
one or more stimulating contacts disposed in the recipient's cochlea configured to open pores in the recipient's cells such that the optically-sensitive elements are transfected into the cells.

17. The stimulating prosthesis of claim 16, wherein the delivery mechanism is configured to introduce optically-sensitive elements configured to, after transfection, facilitate the firing of the cells in the presence of electromagnetic radiation.

18. The stimulating prosthesis of claim 16, wherein the delivery mechanism is configured to introduce optically-sensitive elements configured to, after transfection, inhibit the firing of the cells in the presence of electromagnetic radiation.

19. The stimulating prosthesis of claim 16, wherein the one or more stimulating contacts are optical contacts configured to deliver focused electromagnetic radiation to the cells to open pores in the cells.

20. The stimulating prosthesis of claim 16, wherein the one or more stimulating contacts are electrical contacts configured to deliver an electrical field to the cells to open pores in the cells.
